(19) United States
(12) Patent Application Publication
(10) Pub. No.: US 2016/0270861 A1
(43) Pub. Date: Sep. 22, 2016

(54) SYSTEM AND METHODS FOR A SITUATION AND AWARENESS-BASED INTELLIGENT SURGICAL SYSTEM

(71) Applicant: HEALTH RESEARCH, INC., Buffalo, NY (US)

(72) Inventors: Khurshid Guru, East Amherst, NY (US); Ashirwad Chowriappa, Bangalore (IN)

(21) Appl. No.: 15/032,477

(22) PCT Filed: Oct. 31, 2014
(86) PCT No.: PCT/US14/63595

§ 371 Date: Apr. 27, 2016

Related U.S. Application Data
(60) Provisional application No. 61/898,272, filed on Oct. 31, 2013.

(51) Int. Cl. A61B 34/20; A61B 34/00; A61B 34/32

(52) U.S. Cl. CPC A61B 34/20; A61B 34/32; A61B 34/25; A61B 2034/254; A61B 2034/2055; A61B 2034/2065

(57) ABSTRACT

A computer-based method includes receiving a video feed of a surgical procedure; identifying a current step of the surgical procedure using the video feed and an electronic surgical database; and determining an expected next step of the surgical procedure. A similarity of the expected next step with the actual next step of the surgical procedure may be determined, and an alert and/or surgical guidance may be provided based on the similarity (or lack of). A system for surgical-guidance is disclosed, the system having a storage unit with an electronic surgical database; a surgery image identification unit for analyzing a video feed to determine characteristic data; a mapping unit for matching a database record based on the characteristic data; an alignment unit for mapping matched database records with a stored surgical procedure; and a surgery prediction unit for determining a predicted next surgical step.

### CROSS-REFERENCE TO RELATED APPLICATIONS

This application claims priority to U.S. Provisional Application No. 61/898,272, filed on Oct. 31, 2013, the disclosure of which is incorporated herein by reference.

### FIELD OF THE DISCLOSURE

The disclosure relates to surgical guidance, and in particular to automated surgical guidance by an intelligent system using surgical information stored in an electronic database.

### BACKGROUND OF THE DISCLOSURE

Many surgeries can be broken down into a series of surgical steps performed in a pre-determined order to accomplish the desired result of the surgery. As such, a surgery is also commonly referred to as a surgical procedure. Each of the surgical steps may correspond with one or more surgical activities. For example, a surgical step wherein two anatomical objects are reconnected may involve the surgical activity of suturing the objects with one another.

Medical professionals must often rely on each other to detect errors or provide guidance during a typical surgical procedure. However, outside of an educational setting, it is not common for a sufficiently skilled/educated professional to be available to simply monitor the progress of a surgical procedure. Accordingly, there is a need for an automated system and method which can monitor a surgical procedure and provide guidance to the professionals conducting the procedure.

### BRIEF SUMMARY OF THE DISCLOSURE

A computer-based method for surgical guidance is presented. Embodiments of the method include receiving a video feed of a surgical procedure, the video feed comprising a plurality of image frames; identifying a current step of the surgical procedure based on one or more image frames of the video feed and using an electronic surgical database; and determining an expected next step of the contemporaneous surgical procedure. In some embodiments, identifying a current surgical activity of the surgical procedure based on the one or more image frames of the video feed and using the electronic surgical database; and determining the current step of the surgical procedure based on the identified current surgical activity and the electronic surgical database.

In some embodiments, identifying a current surgical activity includes extracting features of the one or more image frames and matching the extracted features with features of known surgical activities stored in the electronic surgical database.

The method may further comprise determining a similarity of the expected next step with an actual next step of the contemporaneous surgical procedure and providing an alert if the similarity of the expected next step and the actual next step does not exceed a pre-determined threshold. The method may further comprise providing surgical guidance including the expected next step. The surgical guidance may be in the form of audible instruction, visual instruction, a control signal to a robotic system, or other guidance which will be apparent to those having skill in the art in light of the present disclosure. The method may include the step of providing control signals to a robotic system, such as, for example, a surgical robot.

The present disclosure may be embodied as a system for surgical-guidance of a contemporaneous surgery using a video feed of the surgery, comprising a storage unit with an electronic surgical database stored therein; a surgery image identification unit configured to receive the video feed and analyze one or more image frames of the video feed to determine characteristic data; a mapping unit configured to determine a matched database record of the electronic surgical database based on the characteristic data of the surgery image identification unit; an alignment unit configured to map one or more matched database records of the mapping unit with a surgical procedure stored within the electronic surgical database; and a surgery prediction unit for determining a predicted next step of the contemporaneous surgery based on the mapped surgical procedure of the alignment unit.

In another embodiment, a computer-based system for surgical-guidance of a contemporaneous surgical procedure using a video feed of the surgery is provided. Such a system comprises a processor; a communications adapter in electronic communication with the processor and configured to receive the video feed; and a storage medium in electronic communication with the processor and containing an electronic surgical database. The processor is programmed to implement any of the methods disclosed herein. In an exemplary embodiment, the processor is programmed to receive the video feed of a surgical procedure using the communications adapter, the video feed comprising a plurality of image frames; identify a current step of the surgical procedure based on one or more image frames of the video feed and using the electronic surgical database of the storage medium; and determine an expected next step of the contemporaneous surgical procedure.

### DESCRIPTION OF THE DRAWINGS

For a fuller understanding of the nature and objects of the disclosure, reference should be made to the following detailed description taken in conjunction with the accompanying drawings, in which:

FIG. 1 is a diagram depicting a system according to an embodiment of the present disclosure;

FIG. 2 is a diagram depicting a structure of an electronic surgical database for use in an embodiment of the present disclosure;

FIG. 3 is a diagram showing the relationship between surgical procedures, surgical steps, surgical activities, and image frames of a surgical video feed;

FIG. 4 is a diagram of a surgery image identification unit for use in an embodiment of the present disclosure;

FIG. 5 is a diagram of a mapping unit for use in an embodiment of the present disclosure;

FIG. 6 is a diagram of an alignment unit for use in an embodiment of the present disclosure;

FIG. 7 is a diagram of a surgery prediction unit for use in an embodiment of the present disclosure;

FIG. 8 depicts where an image frame (upper left) of a video feed has been matched with six likely matches in an electronic surgical database (image on right side) using the derived feature shown in a plurality of images (lower left);

FIG. 9 is a flowchart of a method according to an embodiment of the present disclosure; and

FIG. 10 is a flowchart of a method according to another embodiment of the present disclosure.

### DETAILED DESCRIPTION OF THE DISCLOSURE

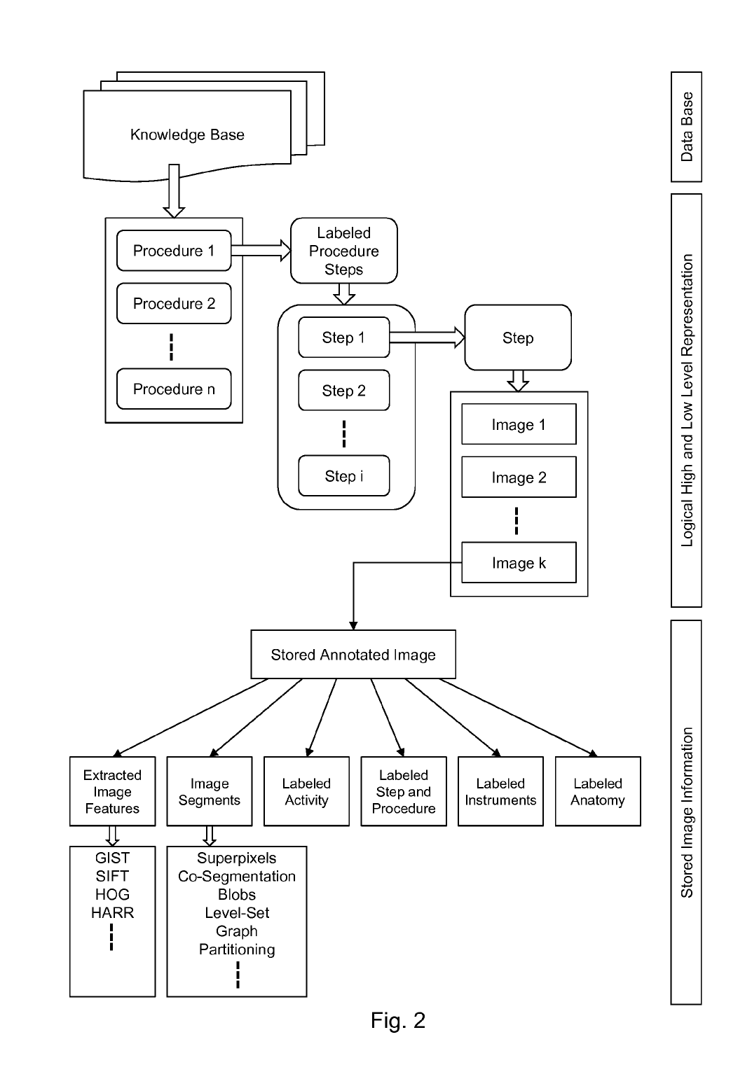

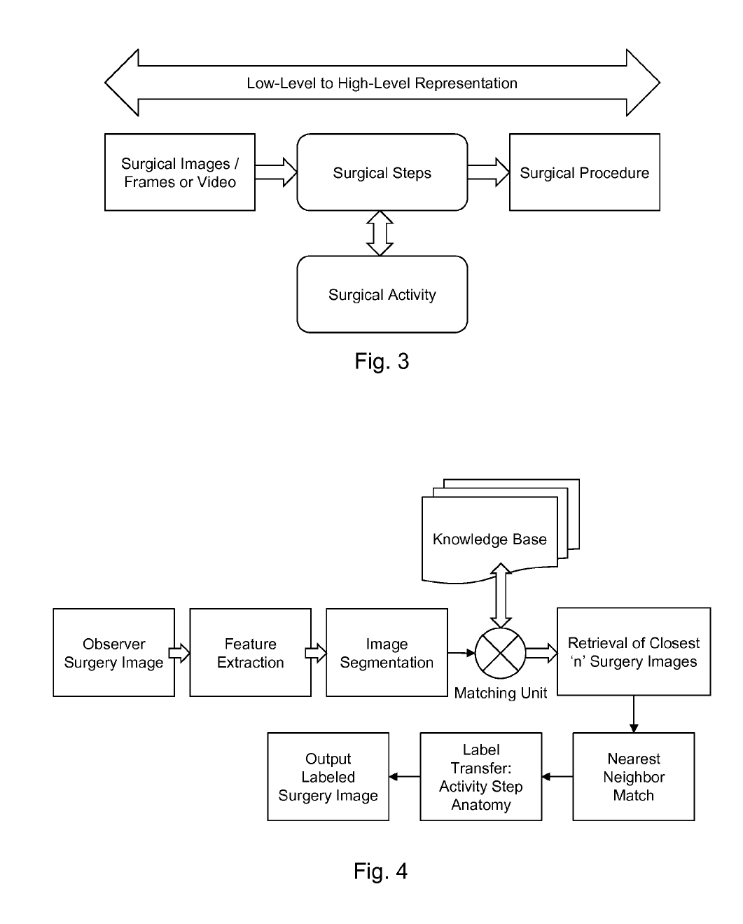

The present disclosure may be embodied as a method 100 for surgical guidance. A computer-based monitoring system is provided with an electronic surgical database, which contains annotated surgical procedure information and match data. The electronic surgical database (FIG. 2) may comprise database records corresponding to surgical procedures, wherein each surgical procedure record may correspond with one or more surgical steps. In the database embodiment depicted in FIG. 2, the surgical steps correspond to one or more stored images each annotated with characteristics such as, for example, extracted image features, image segments, labeled activities, labeled instruments, labeled anatomy, etc.

The method 100 comprises the step of receiving 103 a video feed of a surgical procedure. In an embodiment, the received 103 video feed is of a contemporaneous surgical procedure such that the video feed is received 103 in real-time or near real-time. In some embodiments, near real-time includes a video feed delayed by the latency inherent in the transmission. In other embodiments, the video feed may have delays caused by other factors. The video feed comprises a plurality of image frames. The image frames are generally received consecutively, in chronological order. The video feed may be a two-dimensional (2D) video or a three-dimensional (3D) video. For example, a 3D video feed may comprise a plurality of stereoscopic sets of image frames. For simplicity, embodiments of the present disclosure using stereoscopic sets of image frames (or other formats of 3D image/video information) are included in the present description of 2D image frames. In other words, reference herein to a plurality of image frames in a video feed should be broadly interpreted to include embodiments having a plurality of sets of stereoscopic image frames.

The method 100 comprises identifying 106 a current step of the surgical procedure based on one or more image frames of the received 103 video feed and using the electronic surgical database. In an example, identifying 106 the current step may include analyzing an image frame of the video frame to determine characteristics of the image frame for matching with the match data of the electronic surgical database. Similarly, more than one image frame may be utilized to determine characteristics of the actions in the more than one image frames for matching with the match data. Image-based and action-based techniques are known in the art and one or more such techniques may be used in the present disclosure. For example, feature selection techniques may be used in order to extract features of the image frame(s) and match with extracted features of the match data. Other techniques may include, for example, parametric, nonparametric, geometric, and spectral-based computer vision methods (e.g., template matching, feature selection, clustering, classification, etc.) The location of surgical instruments or patient anatomy may also be used to identify 106 the current step.

To identify 106 the current step, a match probability may be calculated and the resulting surgical step is determined from the annotated surgical information corresponding to the most probable match within the electronic surgical database. Once the current surgical step is identified 106, an expected next step of the surgical procedure may be determined 109 using the electronic surgical database.

In an example, an electronic surgical database may include the surgical steps of a prostatectomy (the surgical procedure). The database contains database records for the surgical steps of the prostatectomy including image and/or action matching information for each step. In an exemplary embodiment of the present method 100, a video feed is received 103 of a contemporaneous prostatectomy. An image frame of the received 103 video feed is analyzed to extract features and the features are compared to the image matching information contained within each database record of the database to determine the most probable match. A database record determined to be the most probable match is used to identify 106 the current surgical step. For example, the image frame may show a dissection taking place, and this image frame is matched to a database record for a dissection surgical step. Once the current surgical step is identified 106, an expected next surgical step can be determined 109 using the electronic database. For example, the matched database record for dissection may include information regarding the next surgical step (e.g., a link to the database record for the next step in the prostatectomy).

The present disclosure is particularly well suited to minimally-invasive surgeries (“MIS”), including, for example, robotically-assisted surgeries, because video feeds are often inherent to such surgeries. Additionally, the point of view of such MIS video feeds is typically the same from surgery to surgery for the same surgical procedure.

The step of identifying 106 a current step of the surgical procedure may comprise the sub-steps of identifying 112 a current surgical activity of the surgical procedure. For example, in the exemplary embodiment of a prostatectomy described above, the surgical step of dissection may correspond to one or more surgical activities such as cutting using a scalpel tool. In such an example, the surgical activity of cutting using a scalpel tool may be identified 112 and the current step of dissection may then be determined 115 based on the identified cutting activity (e.g., in an example where there are no other surgical steps involving the activity of cutting using a scalpel).

The step of identifying 106 a current step of the surgical procedure may be repeated and the resulting plurality of steps may be aligned and mapped 118 to a surgical procedure stored in the electronic surgical database. In this way, a surgical procedure may be identified where it is not known a priori.

The step of identifying a current surgical step may further comprise extracting features of the one or more image frames of the video feed. Such feature extraction techniques are known in the art of computer vision. The extracted features may be probabilistically matched to one or more database records of the electronic surgical database. The database records may comprise images (annotated, labeled, or otherwise identified). In other embodiments, the database records may store extracted features instead of, or in addition to, stored images. In this way, feature extraction does not need to be performed on the stored images at the time of matching with the image frames of the video feed. Instead, the pre-determined and stored features may be matched more efficiently.

The method 100 of the present disclosure may further comprise the step of determining 140 a similarity of the expected next step with an actual next step of the contemporaneous surgical procedure (as identified using the video feed). The determined 140 similarity may be used to provide 143 specific guidance for the surgeon or other medical professional. For example, where the determined 140 similarity is low, an alert may be provided 146 (e.g., audible, visual, tactile, or combination, etc.) In this way, errors in the surgical procedure may be identified and corrected before causing harm.

In other embodiments, the determined 140 similarity may be used to provide qualitative feedback on the surgical procedure. For example, the method 100 may be used in an educational/instructional setting to provide 149 feedback on how well one or more steps of the surgical procedure were performed based on the determined 140 similarity to the steps modeled in the electronic surgical database.

In other embodiments, the system may be used to provide control signals to a surgical robot. The determined 140 similarity may be used to provide 152 control signals to a surgical robot. For example, the method may be used to stop the robotic instruments, or stop the operator on the maser console, or move the robot instruments to a specified location.

In other embodiments, the determined 140 similarity may be used to halt 155 the surgery until corrective measures can take place. For example, in a robotically-assisted surgery, the master console may be disconnected from the slave(s) such that the operator at the master console can no longer move the slave(s).

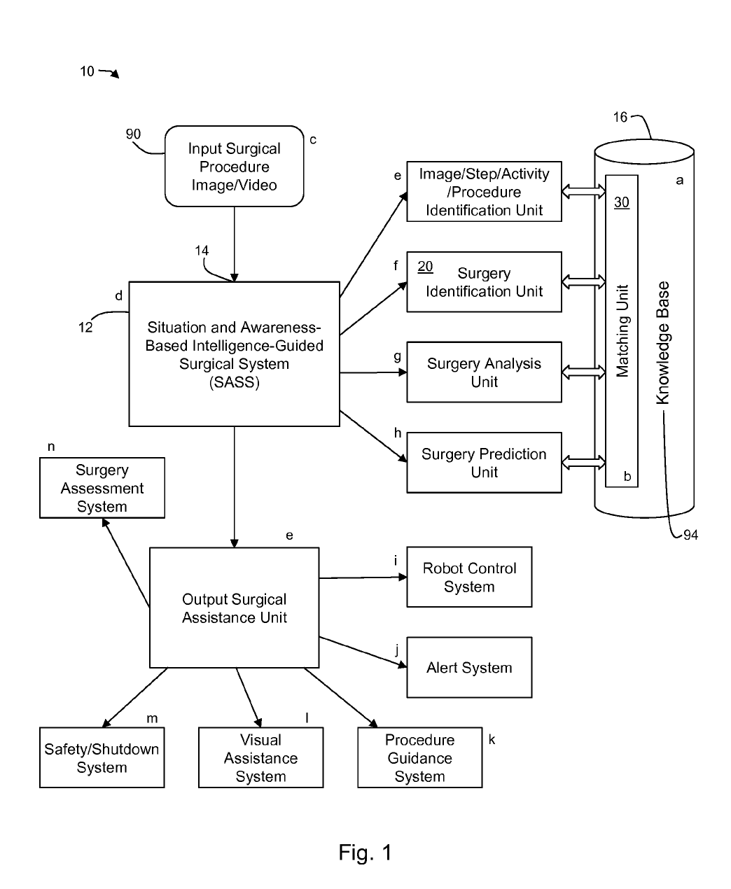

The present disclosure may be embodied as a system 10 for providing surgical guidance. Such a system 10 can be referred to as a “Situation and Awareness-based Intelligence-Guided Surgical System” or “SASS.” With reference to FIG. 1, the system 10 comprises a processing unit 12, which may be, for example, a computer. The processing unit 12 comprises a communications adapter 14 configure to receive a video feed 90. For example, the communications adapter 14 may be a network card for connection to a computer network, and the video feed 90 is received using a communication protocol over the computer network. The processing unit 12 is in electronic communication with a storage unit 16 wherein an electronic surgical database 94 is stored. The storage unit 16 may be a part of the processing unit 12 or separate from the processing unit 12 (e.g., and in communication with the processing unit 12 by way of the communications adapter 14).

The system 10 further comprises a surgery image identification unit 20 (FIG. 4). The surgery image identification unit 20 may form a part of the processing unit 12 or may be separate from the processing unit 12 (an in electronic communication with the processing unit 12). The surgery image identification unit 20 is configured to analyze the video feed 90 received by the processing unit 12 to determined characteristics of one or more image frames of the video feed 90 according to computer vision techniques. For example, the surgery image identification unit 20 may be configured to perform a feature extraction on one or more image frames. The surgery image identification unit 20 may be further configured to segment the image(s).

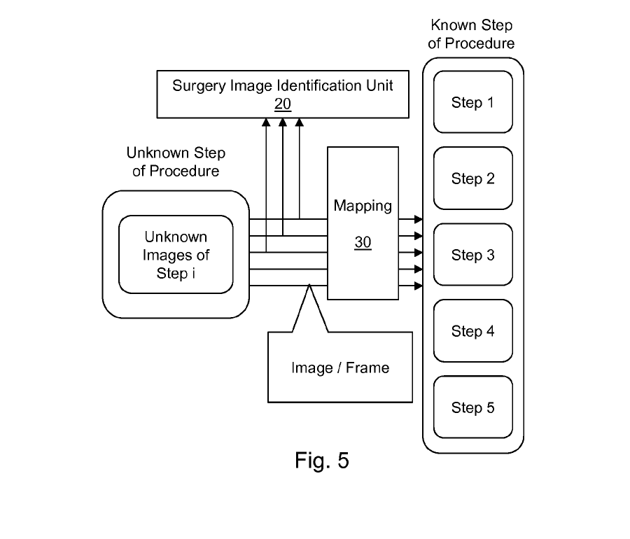

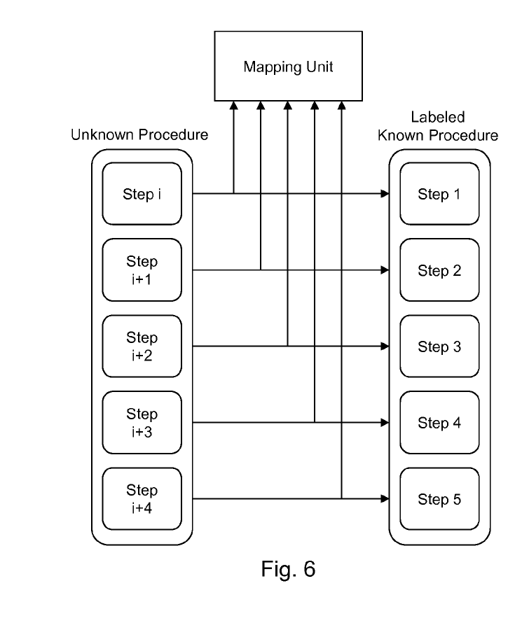

The system 10 further comprises a mapping unit 30 (FIG. 5). The mapping unit 30 may form a part of the processing unit 12 or may be separate from the processing unit 12 (an in electronic communication with the processing unit 12). The mapping unit 30 is configured to compare the analysis data of the surgery image identification unit 20 to the corresponding data of one or more database records in the electronic surgical database 94. For example, the mapping unit 30 may provide a match probability for a match between the analysis data and the one or more database records.

The system 10 (for example, in the surgery image identification unit 20) is further configured to identify database record which is the match of the analysis data and to associate a label of the identified database record with the images which were analyzed (described above). The label may be of a surgical step, a surgical activity, and/or a portion of the anatomy.

The system 10 may be configured to repeat the above analysis for one or more additional image frames of the video feed 90. The analyzed image frames may be consecutive to the previously analyzed image frames, overlapping with the previously analyzed image frames, or separate from (over time) the previously analyzed image frames.

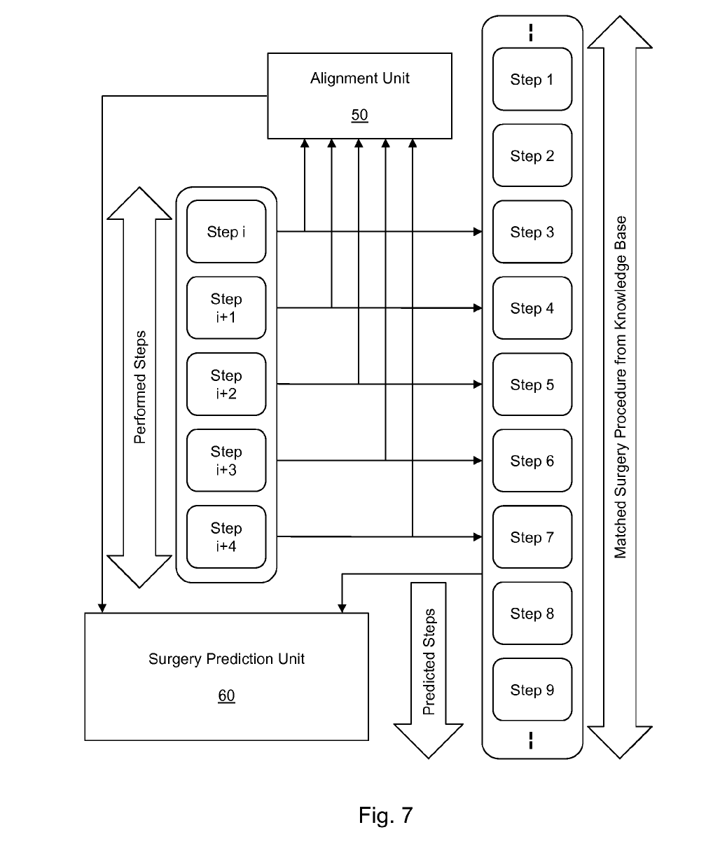

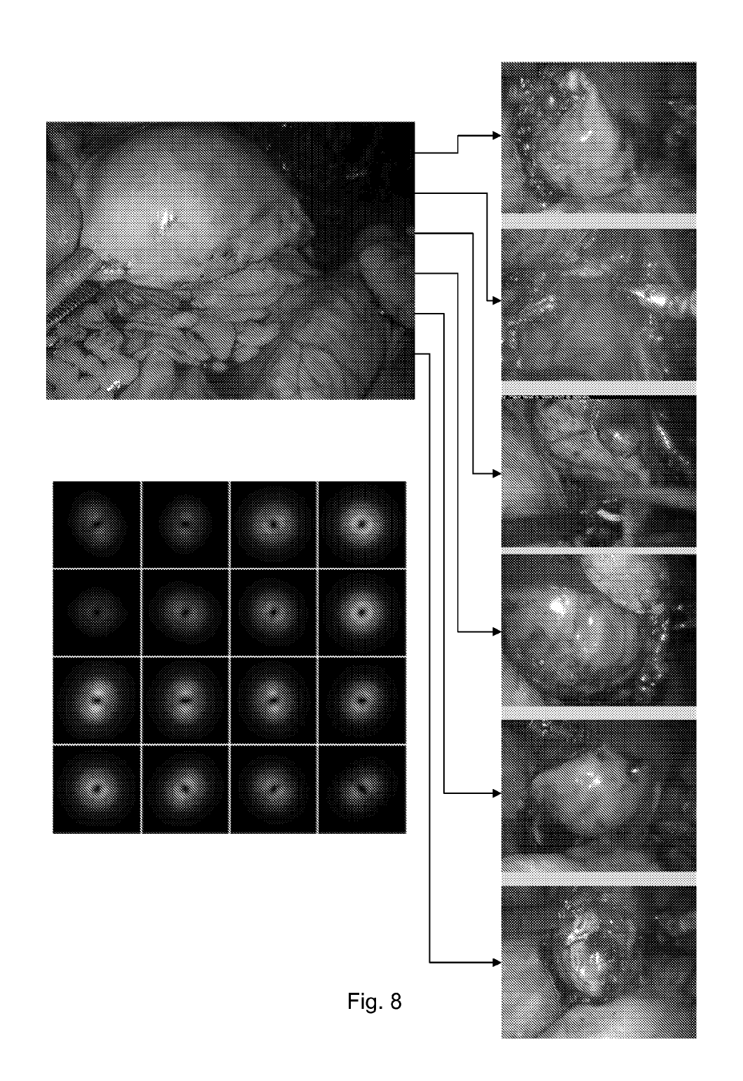

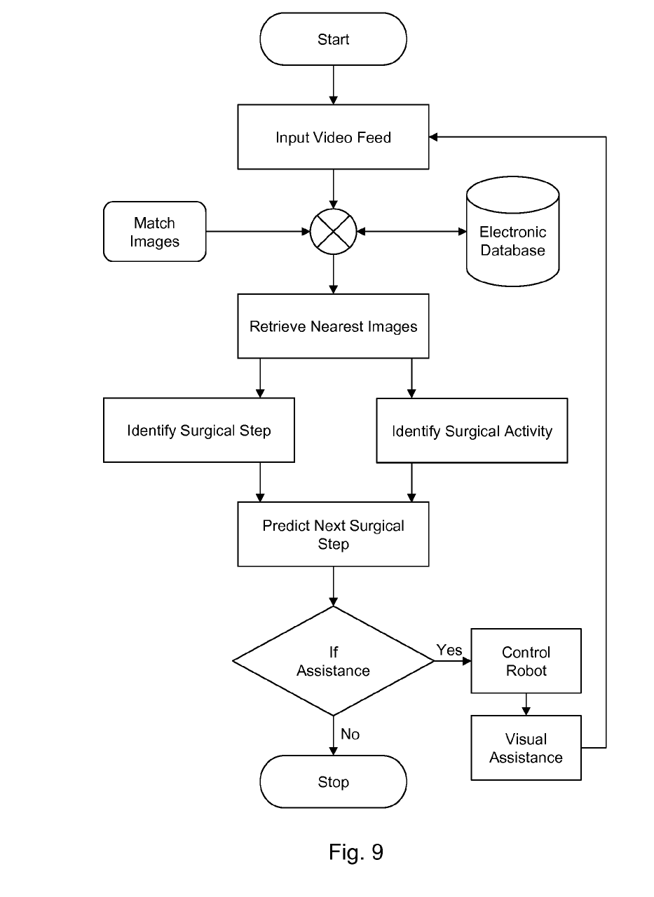

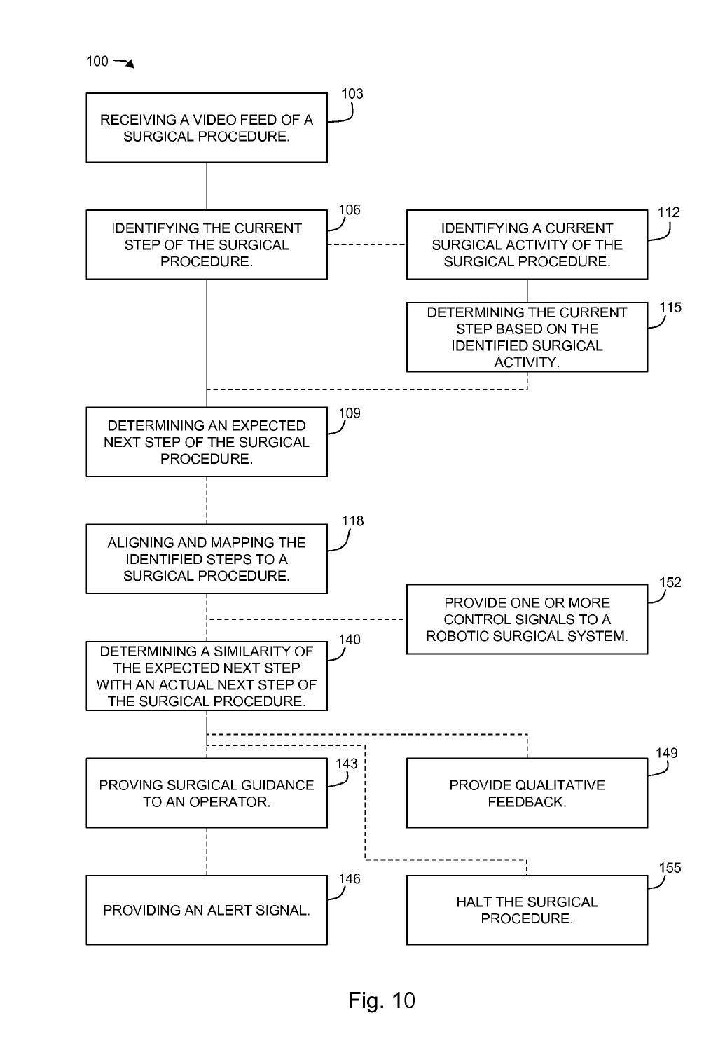

The system 10 may further comprise an alignment unit 50 (FIG. 7), which may form a part of the processing unit 12 or may be separate from the processing unit 12. The alignment unit 50 is configured to receive the labeled surgical steps resulting from the repeated analysis of image frame(s) and map the labeled steps (and the associated order of such labeled steps) with a known surgical procedure contained in the electronic surgical database. For example, if the analyzed and labeled image frames correspond with the surgical steps of dissection, extraction/removal, connection/suturing, the surgical procedure may be identified as a prostatectomy (of course, this example is greatly simplified for convenience).

The system 10 may further comprise a surgery prediction unit 60 (FIG. 7), which may form a part of the processing unit 12 or may be separate from the processing unit 12. The surgery prediction unit 60 is configured to receive surgical procedure information from the alignment unit 50 and labeled surgical information from, for example, the surgery image identification unit 20, and determine, using the electronic surgical database 94, a predicted next surgical step.

The system 10 may be configured to perform any of the disclosed methods to provide surgical guidance. It is to be appreciated that the processor unit 12, the surgery image identification unit 20, the mapping unit 30, the alignment unit 50, and/or the surgery prediction unit 60 may be implemented in practice by any combination of hardware, software, and firmware. Where a unit is implemented in software, the associated program code or instructions may be stored in a processor-readable, non-transitory storage medium, such as a memory.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. A computer-based method for surgical guidance, the method comprising:
receiving a video feed of a surgical procedure, the video feed comprising a plurality of image frames;
identifying a current step of the surgical procedure based on one or more image frames of the video feed and using an electronic surgical database; and
determining an expected next step of the contemporaneous surgical procedure.

2. The method of claim 1, wherein the step of identifying a current step of the surgical procedure comprises:
identifying a current surgical activity of the surgical procedure based on the one or more image frames of the video feed and using the electronic surgical database; and
determining the current step of the surgical procedure based on the identified current surgical activity and the electronic surgical database.

3. The method of claim 1, wherein the step of identifying a current surgical activity comprises:
extracting features of the one or more image frames; and
matching the extracted features with features of known surgical activities stored in the electronic surgical database.

4. The method of claim 1, further comprising:
determining a similarity of the expected next step with an actual next step of the contemporaneous surgical procedure; and
providing an alert if the similarity of the expected next step and the actual next step does not exceed a pre-determined threshold.

5. The method of claim 1, further comprising providing surgical guidance comprising the expected next step.

6. The method of claim 5, wherein the surgical guidance is audible instruction.

7. The method of claim 5, wherein the surgical guidance is visual instruction.

8. The method of claim 5, wherein the surgical guidance is control signal to a robotic system.

9. The method of claim 1, further comprising providing one or more control signals to a robotic system.

10. A system for surgical-guidance of a contemporaneous surgery using a video feed of the surgery, comprising:
a storage unit comprising an electronic surgical database stored therein;
a surgery image identification unit configured to receive the video feed and analyze one or more image frames of the video feed to determine characteristic data;
a mapping unit configured to determine a matched database record of the electronic surgical database based on the characteristic data of the surgery image identification unit;
an alignment unit configured to map one or more matched database records of the mapping unit with a surgical procedure stored within the electronic surgical database; and
a surgery prediction unit for determining a predicted next step of the contemporaneous surgery based on the mapped surgical procedure of the alignment unit.

11. A computer-based system for surgical-guidance of a contemporaneous surgical procedure using a video feed of the surgery, comprising:
a processor;
a communications adapter in electronic communication with the processor and configured to receive the video feed;
a storage medium in electronic communication with the processor and containing an electronic surgical database; and
wherein the processor is programmed to:
receive the video feed of a surgical procedure using the communications adapter, the video feed comprising a plurality of image frames;
identify a current step of the surgical procedure based on one or more image frames of the video feed and using the electronic surgical database of the storage medium; and
determine an expected next step of the contemporaneous surgical procedure.
